SEQUENCE LISTING (3 sequences)

SEQ ID NO: 1 - DNA, 624 bp, Wickerhamomyces ciferrii
  source 1..624: mol_type = genomic DNA
aacaaggtttccgtaggtgaacctgcggaaggatcattatagtattctattgccagcgcttaattgcgcggcgataaaccttacacacactgtttattctttatgaactttgctttgggttgagcctagcttagcccagaggttataaacacattatttttctaatgttttaaacaattaaacctttagtcatgaaattttttataaaataaaaatcttcaaaactttcaacaacggatctcttggttctcgcaacgatgaagaacgcagcgaaatgcgatatgtattgtgaattgcagattttcgtgaatcatcgaatctttgaacgcacattgcgccctctggtattccggagggcatgcctgtttgagcgtcatttctctctcaaacctttgggtttggtattgagtgatactctgtcatatgggttaacttgaaatattgaacagcaagagtgtatttgttagtactccttctgaaataatgtattaggttcttccaactcgttatatcagttaggcagttgcttcggatattttaggctcggcttaataacaataacataaagtttgacctcaaatcaggtaggattacccgctgaacttaagcatatcaataagcgga

SEQ ID NO: 2 - DNA, 19 bp, Synthetic construct
  source 1..19: mol_type = other DNA
tccgtaggtgaacctgcgg

SEQ ID NO: 3 - DNA, 20 bp, Synthetic construct
  source 1..20: mol_type = other DNA
tcctccgcttattgatatgc

(19) United States
(12) Patent Application Publication
(10) Pub. No.: US 2026/0193592 A1
(43) Pub. Date: Jul. 9, 2026

(54) WICKERHAMOMYCES CIFERRII STRAIN HAVING ENHANCED TAPS PRODUCTIVITY AND METHOD OF PRODUCING DESIRED SUBSTANCE USING SAME

(71) Applicant: CRODA KOREA LTD, Iksan-si, Jeonbuk-do (KR)

(72) Inventors: Juhee LEE, Iksan-si, Jeonbuk-do (KR); Kyunghan LEE, Iksan-si, Jeonbuk-do (KR); Hyejin KIM, Iksan-si, Jeonbuk-do (KR)

(73) Assignee: CRODA KOREA LTD, Iksan-si, Jeonbuk-do (KR)

(21) Appl. No.: 19/130,775

(22) PCT Filed: Dec. 22, 2023
(86) PCT No.: PCT/KR2023/021374

§ 371 Date: May 16, 2025

(30) Foreign Application Priority Data
Dec. 30, 2022 (KR) 10-2022-0190563

(51) Int. Cl. C12N 1/145; C12P 7/6436; C12P 13/02; C12R 1/645

(52) U.S. Cl. CPC C12N 1/145; C12P 7/6436; C12P 13/02; C12R 2001/645

(57) ABSTRACT

The present disclosure relates to a Wickerhamomyces ciferrii strain having enhanced tetraacetylphytosphingosine (TAPS) productivity and a method of producing a desired substance using the same. According to the strain according to one aspect, the productivity of sphingolipids and sphingoid bases increases, resulting in an effect of efficiently producing sphingolipids and sphingoid bases through a biological method.

### TECHNICAL FIELD

The present disclosure relates to a novel microorganism with enhanced tetraacetylphytosphingosine (TAPS) productivity, a lysate thereof, a culture medium, an extract of the culture medium, and use thereof.

### BACKGROUND OF THE INVENTION

Sphingolipids refer to a group of lipids derived from sphingoid bases, and are a common name for ceramides or ceramide derivatives that have sphingosine, phytosphingosine, and sphinganine as the basic backbone in the human body. Naturally, sphingolipids are present in a large amount in the cell membranes of animals, plants, and microorganisms, and are involved in various cell signaling such as cell differentiation, cell growth, and cell death.

Ceramides have both hydrophilic groups and lipophilic groups, and thus can not only prevent evaporation of moisture from the skin, but also act as a defense material with permeability of the stratum corneum of the human skin and homeostasis of microorganisms. Recently, ceramides have been used as useful substances in cosmetics.

Tetraacetylphytosphingosine (TAPS), which is a ceramide precursor, is a substance produced in a completely acetylated state by N- and O-acetylation of phytosphingosine among the sphingoid bases, and can be easily converted back to phytosphingosine through a deacetylation reaction. Since the chemical structure of the converted phytosphingosine, has a d-erythro isomer structure which is the same as the ceramide structure of the human skin, TAPS can be used as a raw material available for producing human skin-friendly ceramides.

TAPS can be chemically synthesized (Current Organic Chemistry, 2010. 14 (20) 2483-2521), but the process is complicated and the production cost is high. Therefore, as a way to replace such a process from an economic point of view, TAPS production through a biological method is attracting attention.

### SUMMARY OF THE INVENTION

One aspect is to provide a strain of Wickerhamomyces ciferrii.

The strain may be deposited with the Accession No. KCCM13438P.

Another aspect is to provide a method of producing sphingoid bases or sphingolipids, the method including: culturing the strain in a medium; and isolating sphingoid bases or sphingolipids from the cultured strain, the medium, or a mixture thereof.

Another aspect is to provide use of the strain for producing sphingoid bases or sphingolipids.

### BRIEF DESCRIPTION OF THE DRAWINGS

FIG. 1 is a graph comparing and analyzing production yields of tetraacetylphytosphingosine (TAPS) by strains according to an embodiment.

FIG. 2 is a graph comparing and analyzing the growth of strains according to an embodiment.

FIG. 3 is a graph comparing and analyzing the consumption amount of glycerol by strains according to an embodiment.

### DETAILED DESCRIPTION OF THE INVENTION

One aspect provides a Wickerhamomyces ciferrii strain.

In an embodiment, the strain may be deposited with the Accession No. KCCM13438P.

In an embodiment, the W. ciferrii strain may be a strain including 16S rRNA having a nucleotide sequence of SEQ ID NO: 1.

In an embodiment, the strain may be a strain including 16S rRNA consisting of a nucleotide sequence of SEQ ID NO: 1 or 16S rRNA having a nucleotide sequence with nucleotide sequence homology of at least 97% to the nucleotide sequence of SEQ ID NO: 1. In detail, the nucleotide sequence of may have at least 93%, 95%, 96%, 97%, 98%, 99%, 99.5%, 99.8%, 99.9%, or 100% homology to the nucleotide sequence of SEQ ID NO: 1 of the present specification.

In an embodiment, the strain may include a naturally occurring mutation of the W. ciferrii strain.

Throughout the specification, the terms “a mutant, a variant, and a genetically altered mutant” include strains of W. ciferrii of which genetic and/or phenotypic properties are changed compared to mother cells or wild-type cells. A naturally occurring variant of the W. ciferrii strain may include an automatic alteration of selectively isolated desired properties. A genetic variation includes introduction of exogenous and/or endogenous DNA sequences into the genome of the W. ciferrii strain by insertion into the genome of the strain by means of a delivery vehicle (e.g., a vector).

A naturally occurring or artificially induced mutation includes, as a result of alterations in the amino acid sequences encoded by the DNA sequences, at least single nucleotide alteration such as deletion, insertion, translocation, or other DNA displacement.

Also, the terms “a mutation, a variant, and a genetically altered mutant” may include: variants that are not achieved by predetermined (in vitro) promotion of the genome, but provide a benefit of selection to support the cell survival when exposed to environmental pressures such as antibiotics; and/or natural mutations acquired through natural selection of mutants; and/or any micro-organisms that arise through the gain and/or loss of genes; and/or strains of W. ciferrii that have undergone genetic alterations accumulating in the genome at rates that are virtually identical to genetic alterations.

It is understood by those skilled in the art that mutant or variant strains of W. ciferrii can be identified by DNA sequence homology analysis with parent cells. The W. ciferrii strain having sequence identity close to the parent cells may be considered to be mutant or variant strains. The W. ciferrii strain with at least 96% sequence identity (homology), such as at least 97%, at least 98%, or at least 99% sequence identity (homology), to the parent DNA sequences may be considered to be a mutant or variant strain. The sequence homology can be determined by using the online homology algorithm “BLAST” program.

In some embodiments, the strain may have improved productivity of sphingoid bases and sphingolipids, for example, tetraacetylphytosphingosine (TAPS), compared to the parent cells or wild-type cells.

The term “parent cells” is used interchangeably with wild-type cells and original cells, and may refer to a starting material for screening the strain according to an embodiment.

The strain according to an embodiment may have increased activity of enzymes that are involved in biosynthetic pathways of sphingoid bases and/or sphingolipids, compared to the parent cells. Examples of known enzymes that are involved in biosynthetic pathways of sphingoid bases and/or sphingolipids may include an acetyl group transferase, a ceramide synthetase, or a sphingolipid desaturase.

The term “increase in activity” or “increased activity” as used in the present specification may refer to a detectable increase in the activity of cells, proteins, or enzymes. The “increase in activity” or “increased activity” may refer to the activity of modified cells, modified proteins, or modified enzymes at a higher level than the activity of cells (e.g., parent cells), proteins, or enzymes of the same type to be compared. The term “cell activity” may refer to the activity of a specific protein or enzyme in a cell. Cells having increased activity may be cells having alterations with increased activity of one or more enzymes or polypeptides compared to parent cells.

Another aspect provides a lysate, a fermentation broth, a culture medium, or a mixture thereof of the strain.

The term “culture medium” or “fermentation product” as used in the present specification may be used interchangeably with “supernatant of culture or fermentation”, “supernatant of culture product or fermentation product”, “conditioned culture medium or fermentation broth”, or “conditioned medium”, and may refer to a whole medium including the strain, metabolites thereof, extra nutrients, etc., wherein the strain can be obtained by culturing the strain for a certain period of time in a medium capable of supplying nutrients for the growth and survival of the strain in vitro.

The culture medium or fermentation product may be: a culture medium or fermentation broth itself obtained by culturing or fermenting the strain, a concentrate thereof, or a freeze-dried product thereof; or a culture supernatant or fermentation supernatant obtained by removing the strain from a culture medium or fermentation broth, a concentrate thereof, or a freeze-dried product thereof.

The term “lysate” as used in the present specification may be used interchangeably with “dissolved product”, and refer to a solution or suspension in an aqueous medium containing broken cells. Cell lysates include, for example, macromolecules such as DNA, RNA, proteins, peptides, carbohydrates, lipids, etc., and/or micromolecules such as amino acids, sugars, fatty acids, etc., or fractions thereof.

Another aspect provides a method of producing sphingoid bases or sphingolipids, the method including: culturing a strain of Wickerhamomyces ciferrii in a medium; and isolating sphingoid bases or sphingolipids from the strain cultured, the medium, or a mixture thereof.

Another aspect provides use of a strain of Wickerhamomyces ciferrii for the production of sphingoid bases or sphingolipids.

The term “sphingoid base” in the context of the present specification may refer to phytosphingosine, sphingosine, sphingadienine, 6-hydroxysphingosine, and sphinganine (dihydrosphingosine), and also include those in an acetylated form, such as tetraacetylphytosphingosine, triacetylphytosphingosine, diacetylphytosphingosine, O-acetylphytosphingosine, triacetylsphinganine, diacetylsphinganine, O-acetylsphinganine, triacetylsphingosine, diacetylsphingosine, O-acetylsphingosine, tetraacetyl-6-hydroxysphingosine, triacetyl-6-hydroxysphingosine, diacetyl-6-hydroxysphingosine, O-acetyl-6-hydroxysphingadienine, triacetylsphingadienine, diacetylsphingadienine, or O-acetylsphingadienine.

The term “sphingolipid” in the context of the present specification may refer to a compound including a sphingoid base covalently bound to a fatty acid via an amide bond. Fatty acids may be saturated or mono- or polyunsaturated. The side chains of fatty acids may vary in length. The side chains of fatty acids may also include functional groups such as hydroxyl groups. Examples of sphingolipids may include phytoceramides, ceramides, and dihydroceramides, and more complicated glucosylceramides (cerebrosides) and inositol phosphorylceramides, mannosyl-inositol phosphorylceramides and mannosyl-diinositol phosphorylceramides. Here, sphingolipids may include sphingoid bases bonded to acetyl radicals via amide bonds, such as N-acetylphytosphingosine, N-acetylsphinganine, N-acetylsphingosine, or N-acetyl-6-hydroxysphingosine.

The use of cells according to the present specification for producing sphingoid bases and sphingolipids selected from the group consisting of phytosphingosine, sphingosine, sphingadienine, 6-hydroxysphingosine, sphinganine tetraacetylphytosphingosine (TAPS), (dihydrosphingosine), triacetylphytosphingosine, diacetyl Phytosphingosine, O-acetylsphingosine, N-acetylsphingosine, triacetylsphinganine (TriASa), diacetylsphinganine, O-acetylsphinganine, N-acetylsphinganine, Triacetylsphingosine (TriASo), diacetylsphingosine, O-acetylsphingosine, N-acetylsphingosine, tetraacetyl-6-triacetyl-6-hydroxysphingosine, diacetyl-6-hydroxysphingosine, hydroxysphingosine, O-acetyl-6-hydroxysphingosine, N-acetyl-6-hydroxysphingosine, triacetylsphingadienine, diacetylsphingadienine, and O-acetylsphingadienine is particularly advantageous. Also, the use of the cells according to the present specification for producing TAPS is particularly preferable.

Carbon sources that can be used in the cell culturing may be carbohydrates, such as glucose, fructose, glycerol, sucrose, maltose, molasses, or other alcohols such as ethanol, and organic acids such as acetate. Nitrogen sources that can be used may be, for example, ammonia, ammonium sulfate, ammonium nitrate, ammonium chloride, or organic nitrogen compounds (e.g., yeast extract, malt extract, peptone, corn steep liquor). Inorganic compounds such as phosphate salts, magnesium salts, potassium salts, zinc salts, iron salts, and the like may also be used.

In an embodiment, the culturing step may include culturing under conditions enabling the production of sphingoid bases and sphingolipids (also, under conditions favorable for the production of TAPS).

When the sphingoid bases and the sphingolipids are TAPS, the method may further include deacetylating the obtained TAPS.

Suitable culture or fermentation conditions for the strain of the present disclosure are known to those skilled in the art, for example, from International Publication No. 2007-131720 or Korean Patent Registration No. 10-0221357, and these documents are incorporated herein by reference.

In detail, fermentation or culturing may be performed over a period of 0.5 days to 30 days. Fermentation or culturing may suitably be a batch process or a continuous or fed-batch process, at a temperature in a range of about 0 to 45° C. and a pH of 2 to 10. Preferred fermentation or culture conditions may include a temperature in a range of about 20° C. to about 37° C. and/or a pH of 3 to 9. Appropriate conditions may generally be selected based on the choice of an expression host and a desired material to be produced.

After fermentation or culture, as necessary, the cells may be removed from the fermentation medium or culture medium by centrifugation or filtration. Then, a desired material of the present specification, for example, sphingoid bases or sphingolipids, may be collected, and purified and isolated by conventional means.

According to the novel strain according to an aspect, and a lysate derived therefrom, a culture broth, an extract of the culture broth, or a mixture thereof, the productivity of sphingolipids and sphingoid bases is increased, resulting in an effect of efficiently producing sphingolipids and sphingoid bases through biological methods.

### EXAMPLES

Hereinafter, preferable Examples are presented to help understanding of the present disclosure. However, Examples below are only presented for easier understanding of the present disclosure, and the contents of the present disclosure are not limited by the following examples. Since Examples can apply various modifications, Examples are not limited to those disclosed below and can be implemented in various forms.

### Example 1. Isolation and Identification of Wickerhamomyces ciferrii Strain

### 1.1. Isolation of Strain

A W. ciferrii SB8-25 strain of the present disclosure was isolated from new single spore derivatives derived from the W. ciferrii KCCM 50215 (hereinafter also referred to as KCCM 50215) which was a parent strain.

Specifically, the KCCM 50215, which was diploid, was shaking-cultured in cultured with shaking at 25° C. for 3 days in YMgl medium (supplemented with 0.3% of yeast extract, 0.3% of malt extract, 0.5% of petone, 3% of glycerol). Afterwards, 0.1 ml to 0.5 ml of the culture medium was spread and cultured for 7 days to 10 days in a spore-forming plate medium (supplemented with 5% of malt extract and 3% of agar), and formation of hat-shaped spores was observed. To selectively isolate the formed spores, heat shock was applied. The heat shock was treated on a test tube containing 1 ml to 2 ml of spore suspension (6×10⁷ cells/ml to 1×10⁸ cells/ml) at 55° C. for 1 minute to 5 minutes. Then, 0.1 ml of the cells per hour was spread on YMgl plate medium containing 2 mM to 30 mM of EGTA, and cultured at 25° C. First selection was carried out to isolate colonies different from the parental strain in colony types, colors, and sizes from among the formed cell colonies. Second selection was carried out by evaluating the primary selected single spore derivative, in consideration of the cell morphology, degree of precipitation, and degree of hydrophobic films formed on the surface of the culture test tube. The secondary selected single spore derivatives were cultured in YMgl plate medium at 25° C. for 4 days, and then left in a refrigerator at 4° C. for 2 days to 3 days to induce crystallization of sphingolipids secreted out of the cells. Through the microscopic observation, strains with many crystals of sphingolipids were finally selected. In summary, it was observed that the formed crystals were melted when heated in an oven at 80° C. for about 30 minutes, and strains with the highest TAPS production yield was finally selected by thin layer chromatography (TLC) analysis. The quantitative TAPS analysis of the selected strains was carried out by high performance liquid chromatography (HPLC).

Among the single spore derivatives, the W. ciferrii strain with the highest TAPS production yield was selected.

### 1.2. Molecular Biological Identification of Selected Strain

The identification of the finally selected W. ciferrii strain in Example 1.1 was carried out based on the 16S rRNA gene sequence similarity. The strain on the plate isolated in the above example was requested to MACROGEN to analyze the 16S rRNA sequences. Specifically, PCR was performed using the primer sequences shown in Table 1 below.

TABLE 1

| Gene | Primer | Sequence (5′→3′) |
|---|---|---|
| 16S | ITS5 | TCCGTAGGTGAACCTGCGG |
| rRNA | | (SEQ ID NO: 2) |
| | 1TS4 | TCCTCCGCTTATTGATATGC |
| | | (SEQ ID NO: 3) |

The 16S rRNA gene sequence of the strain analyzed using the ITS5 and 1TS4 primers for PCR amplification is expressed as SEQ ID NO: 1.

Accordingly, the strain was named “W. ciferrii SB8-25” (hereinafter also referred to as ‘SB8-25’), and was deposited with the Korean Culture Center of Microorganisms (KCCM) on Sep. 22, 2022, and assigned Accession No. KFCC11942P, which was converted to KCCM13438P on Dec. 13, 2023 for International deposit.

### 1.3. Analysis of Carbon Source Availability of Selected Strain

To confirm the mycological characteristics of the finally selected W. ciferrii in Example 1.1., the availability of each carbon source was analyzed.

Specifically, after inoculating and culturing cupules of a kit each containing the W. ciferrii strain and a unique carbon source, it was determined by turbidity change whether the test bacteria could use these sugars, and the results are shown in Table 2 below.

TABLE 2

| Carbon source | usability |
|---|---|
| D-glucose | ◯ |
| Glycerin | ◯ |
| Ketogluconate | X |
| D-arabinose | X |
| D-xylose | X |
| Adonitol | ◯ |
| Lactose | X |
| Cellobiose | X |
| N-acetylglucosamine | X |
| Methyl-D-glucoside | ◯ |
| Maltose | ◯ |
| Sucrose | ◯ |
| D-galactose | X |
| Inositol | X |
| Xylitol | X |
| Sorbitol | ◯ |
| Trehalose | ◯ |
| Melesitos | X |
| Raffinose | X |

### Experimental Example 1. Analysis of W. ciferrii SB8-25 Strain on TAPS Production Yield

The TAPS productivity of the W. ciferrii SB8-25 strain and the wild-type W. ciferrii strain (Accession No. KCCM 50215) as a control group was compared.

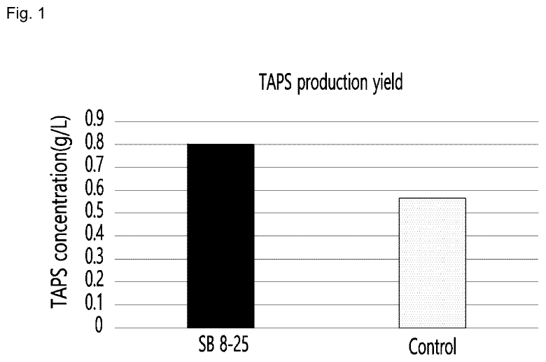

Specifically, the W. ciferrii SB8-25 strain (Accession No: KCCM13438P) and the wild-type W. ciferrii strain (Accession No: KCCM 50215) were each inoculated to 50 ml of YGM optimal medium (supplemented with 100 g/l of glycerol, 2 g/l of yeast extract, 3 g/l of KNO₃, 0.5 g/l of (NH₄)₂SO₄, 0.3 g/l of MgSO₄·7H₂O, 0.5 g/l of NaCl, 3 g/l of corn steep liquor (CSL), and 1 g/l of LS-300 (anti-forming agent), cultured at 25° C. for 5 days at 220 rpm. Then, methanol was added thereto and reacted at room temperature for 1 hour, and TAPS in the supernatant obtained by centrifugation was analyzed. The TAPS production yield was analyzed by HPLC using UV light at 200 nm, and was measured at 24 hours, 48 hours, 72 hours, 96 hours, and 120 hours, respectively. Here, as a solvent, a mixture of methanol:water:trifluoroacetic acid (TFA) at a ratio of 84.5:18.45:0.05 was used. The results are shown in FIG. 1.

FIG. 1 is a graph comparing and analyzing production yields of TAPS by the strain according to an embodiment.

As shown in FIG. 1, compared to the control strain, the highest TAPS production yield was measured in the culture supernatant of the W. ciferrii SB8-25 strain.

### Experimental Example 2. Analysis of W. ciferrii SB8-25 Strain on Growth

The growth of the W. ciferrii SB8-25 strain and the wild-type W. ciferri strain (Accession No. KCCM 50215) as a control group was compared.

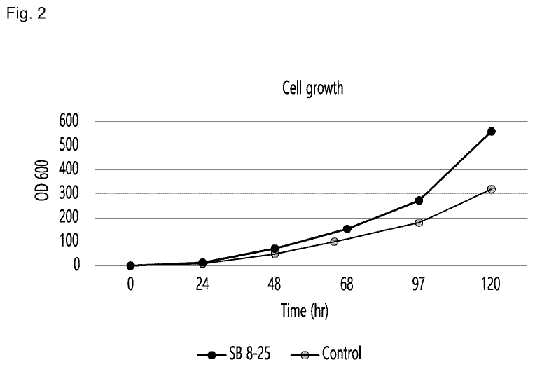

Specifically, each yeast pre-cultured in YMgl medium was inoculated with 100 ml of YMgl to have the same absorbance (O.D. 600=0.05), and shake-cultured at 30° C. and at a speed of 250 rpm. Samples were taken at 0 hours, 24 hours, 48 hours, 68 hours, 97 hours, and 120 hours, respectively, and the absorbance (O.D. 600) was measured. The results are shown in FIG. 2.

FIG. 2 is a graph comparing and analyzing the growth of the strain according to an embodiment.

As shown in FIG. 2, compared to the control strain, it was confirmed that the W. ciferrii SB8-25 strain grew the most after 120 hours of the culturing.

It has been reported that the production of TAPS, an acetylated form of phytosphingosine, was proportional to the amount of cells. Through the above results, in contrast to the control strain, it was confirmed that the W. ciferrii SB8-25 strain had not only improved TAPS yield, but also improved amount of cells.

### Experimental Example 3. Analysis of W. ciferrii SB8-25 Strain on Glycerol Consumption Amount

The glycerol consumption amount of the W. ciferrii SB8-25 strain and the wild-type W. ciferrii strain (Accession No. KCCM 50215) as a control group was compared.

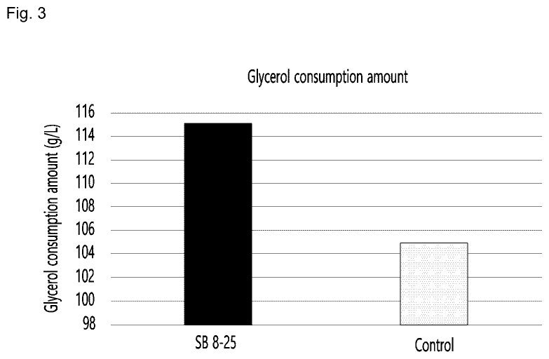

Specifically, the strain was cultured and centrifuged to obtain a supernatant in the same manner as in Experimental Example 1, and the samples were taken at 0 hour, 24 hours, 48 hours, 68 hours, 97 hours, and 120 hours, respectively, to measure the consumption amount of glycerol. The results are shown in FIG. 3.

FIG. 3 is a graph comparing and analyzing the consumption amount of glycerol of the strain according to an embodiment.

As shown in FIG. 3, compared to the control strain, it was confirmed that the W. ciferrii SB8-25 strain had remarkably excellent glycerol consumption amount, that is, glycerol usability.

According to the novel strain according to an aspect, and a lysate derived therefrom, a culture broth, an extract of the culture broth, or a mixture thereof, the productivity of sphingolipids and sphingoid bases is increased, resulting in an effect of efficiently producing sphingolipids and sphingoid bases through biological methods.

The foregoing descriptions are only for illustrating the disclosure, and it will be apparent to a person having ordinary skill in the art to which the present invention pertains that the embodiments disclosed herein can be easily modified into other specific forms without changing the technical spirit or essential features. Therefore, it should be understood that Examples described herein are illustrative in all respects and are not limited.

### ASSESSION NUMBER

Name of Depository Authority: Korean Culture Center of Microorganisms
Accession number: KCCM13438P
Accession date: 20231213

1. A strain of Wickerhamomyces ciferrii, deposited with the Accession No. KCCM13438P.

2. The strain of claim 1, wherein the strain comprises 16S rRNA having a nucleotide sequence of SEQ ID NO: 1.

3. The strain of claim 1, wherein the strain comprises a naturally occurring mutation of the W. ciferrii strain.

4. The strain of claim 1, wherein the strain has enhanced tetraacetyl phytosphingosine (TAPS) productivity compared to a wild-type strain.

5. The strain of claim 1, wherein the strain uses, as a carbon source, at least one selected from the group consisting of D-glucose, glycerin, adonitol, methyl-D-glucoside, maltose, sucrose, sorbitol, and trehalose.

6. A method of producing sphingoid bases or sphingolipids, the method comprising:
culturing the strain of claim 1 in a medium; and
isolating sphingoid bases or sphingolipids from the strain cultured, the medium, or a mixture thereof.

7. The method of claim 6, wherein the medium is a medium containing a carbon source.

8. The method of claim 7, wherein the carbon source is glycerol.

9. The method of claim 6, wherein the culturing is performed under conditions enabling the production of sphingoid bases and sphingolipids.

10. The method of claim 6, wherein the sphingoid bases or the sphingolipids are tetraacetylphytosphingosine (TAPS).

11. The method of claim 9, further comprising, after culturing the strain under conditions favorable for the production of tetraacetylphytosphingosine (TAPS), deacetylating the TAPS thus obtained.
